(19) United States
(12) Patent Application Publication
(10) Pub. No.: US 2003/0031702 A1
(43) Pub. Date: Feb. 13, 2003

(54) Safer teratogenic pharmaceuticals

(76) Inventor: Leonard L. Mazur, Mountain Lakes, NJ (US)

Correspondence Address: PHARMACEUTICAL PATENT ATTORNEYS, POHL & ASSOC. LLC, 55 MADISON AVENUE, 4TH FLOOR (P4014), MORRISTOWN, NJ, 07960-6397, US

(21) Appl. No.: 09/915,467

(22) Filed: Jul. 27, 2001

(51) Int. Cl. A61F 6/06; A61F 13/00

(52) U.S. Cl. 424/430

(57) ABSTRACT

A pharmaceutical product comprising isotretinoin and a contraceptive.

### RELATED APPLICATIONS

None.

### GOVERNMENT INTEREST

None.

### BACKGROUND

Teratogenic pharmaceuticals provide medical benefits, but pose the danger of creating birth defects. I have found a way to make teratogenic pharmaceuticals safer. First, I will briefly discuss one teratogenic pharmaceutical.

Isotretinoin

Retinoic acid, (all-E)-3,7-Dimethyl-9-(2,6,6-trimethyl-1-cyclohexen-1-yl)-2,4,6,8-nonatetraenoic acid, also called vitamin A acid or tretinoin, is the physiological metabolite of retinol. Its preparation is known in the art, Robeson, C. D. et al. , 77 J. Amer. Chem. Soc. 4111 (1955); U.S. Pat. No. 3,006,939 (1961); Lakshmanan et al., 90 Biochem. J. 569 (1964). It has been investigated for use in treating photoaged skin, Weiss, J. S. et al., 259 J. Amer. Med. Assoc. 527 (1988), and in promyelocytic leukemia, Warrell, R. P. et al., 8 Leukemia 929 (1994). See generally Sporn, M. B. et al., The Retinoids (Academic Press, New York 1984).

The 13-cis form may be produced in a single-step process as known in the art. Marbet, R., U.S. Pat. No. 3,746,730 (1973); German Letters Patent No. 2,061,507 (1971). The 13-cis form was found effective in treating severe cystic acne. Peck, G. L. et al., 300 N. Engl. J. Med. 329 (1979). The 13-cis form is thus known as a keratolytic. It is commercially available under the tradename ACCUTANE®. See Invention Disclosure Statement (Accutane® product label).

The teratogenicity of the compound has also been studied. Lammer, E. J. et al., 313 N. Engl. J. Med. 837 (1985). The teratogenicity of the compound is now widely known in the art. E.g., Gaull, U.S. Pat. No. 4,545,977 (1985) at col. 1, lines 21-35.

Patients taking isotretinoin are thus advised to not become pregnant. See Invention Disclosure Statement (Accutane® product label). While this advice is medically correct and appropriate, it can fail in actual practice. For example, from 1982 to 2000, at least two thousand women in the United States taking ACCUTANE® isotretinoin have become pregnant. See Invention Disclosure Statement (Roche Pharma., Inc., Appendix I Pregnancy Data, at Table 1). This number does not include non-United States pregnancies, nor pregnancies not reported (e.g., unreported spontaneous abortions, etc . . . ). It also does not include pregnancies associated with other potentially teratogenic pharmaceuticals (e.g., methotrexate). Thus, there were likely far more than two thousand pregnancies associated with teratogenic pharmaceuticals. Thus, despite ostensibly clear warnings, the risk of unexpected pregnancy remains.

The reason for these pregnancies, despite ostensibly clear admonitions to avoid pregnancy, is unclear. The failure to avoid pregnancy could be due to simple patient misunderstanding, albeit this seems unlikely. Of the reported pregnancies, only 2.8% occur in girls less than 16 years of age, and only 0.27% in girls known to have not completed high school. Id. at Tables 4-5. The other patients would likely be able to comprehend and apprehend the warnings given.

It could be because patients forget the admonition to not become pregnant. Dr. Janet Woodcock, the Director of the United States Food & Drug Administration Center for Drug Evaluation and Research, noted, “exclusive reliance on ‘human memory’ is not an adequate precaution for managing severe risks.” See Invention Disclosure Statement at Woodcock, J., Accutane Letter (Jan. 9, 2001). She concludes that “additional systematized measures to manage risk and fully inform patients and families should be instituted, given the devastating impact of potential side effects.” Id.

To provide such systematized measures, Elsayed et al., U.S. Pat. No. 6,045,501, discloses using a computer software based patient registry. This registry ostensibly identifies and catalogs patients who are taking a teratogenic pharmaceutical, to more readily identify those patients who are at risk of becoming pregnant. In so doing, it is easier to contact these patients by, for example, telephone, to remind them to not become pregnant.

Another approach entails avoiding the use of the potential teratogen altogether. For example, isotretinoin is expressly labeled for use only after other, non-teratogenic alternative therapies have failed. Treating acne may be attempted by using not isotretinoin, but other compounds such as antibiotics, silver compounds, and other compositions. See Invention Disclosure Statement at U.S. Pat. Nos. 5,260,292; 5,439,923; 5,455,262; 5,470,834; 5,688,812; 5,744,151; 5,837,270; 5,914,340; 5,962,517; 6,008,254. Such alternatives meet with only mixed clinical success. Thus, in certain cases, using the potentially teratogenic pharmaceutical is unavoidable.

Another approach, disclosed in Gaull, U.S. Pat. No. 4,545,977, entails administering taurine with isotretinoin. Gaull asserts that taurine reduces the teratogenic effect of isotretinoin. While this may work, I do not at this point have conviction that it works reliably enough to eliminate the the risk altogether.

Thus, there remains a need for a way to make the administration of isotretinoin and other teratogenic pharmaceuticals safer. I have found a way.

### SUMMARY

I have found that one can make teratogenic pharmaceuticals more safe, by combining them as a unit with a contraceptive. This combination improves the ability to assure that the patient fully understands that avoiding pregnancy is a mandatory and continuing part of the treatment with the teratogenic pharmaceutical, and thus reduces the risk of unwanted pregnancy. In so doing, it minimizes the risk that a patient will become pregnant while taking the teratogen.

Such co-administration of a contraceptive compound has often been discouraged by the art, even while the art acknowledges pregnancy should be avoided. This teaching against co-administration of a contraceptive may be due to concerns over cross-reactivity between the contraceptive and the teratogen. For example, Shangold et al., U.S. Pat. No. 6,214,815, disclose a contraceptive pharmaceutical. Shangold expressly teaches, however, to not use contraceptives where there is “concomitant use of isotretinoin (Accutane), tretinoin (Renova or Retin-A) or has taken them within the 30 day period immediately prior to the screening visit.” Id. at col. 11, lines 49-54.

### DETAILED DESCRIPTION

Contraceptives

I use the term “contraceptive” herein to mean something that prevents or inhibits conception. This can be, for example, a device such as a condom or an intrauterine device. It can alternatively (or additionally) be a pharmaceutical in an amount effective to act as a birth control pharmaceutical.

Note that the claims require both a teratogen and a contraceptive; thus, the claim term “teratogenic pharmaceutical” excludes the same chemical used as a contraceptive pharmaceutical. For example, increased risk of birth defects (teratogenicity) is listed as a side effect on several commercially available contraceptive products, including ENCARE® vaginal contraceptive suppositories (commercially available from Blairex Pharmaceutical Corp.); TRIPHASIL®, OVRAL®, LO/OVRAL®, OVRETTE®, NORDETTE®, PREMPHASE® and PREMPRO® tablets, and PREMARIN® intravenous and vaginal cream (each commercially available from the Wyeth-Ayerst Pharmaceutical division of American Home Products Corp.); and Ortho DIENESTROL® cream (commercially available from Ortho-McNeil Pharmaceutical Corp.). With these products, the claims require more than the contraceptive product alone; the claims require the product combined with another teratogen.

Teratogenic Pharmaceuticals

I use the term “teratogenic” to include pharmaceuticals associated with an increased risk of birth defects. The term thus includes pharmaceuticals with FDA-approved labeling citing an increased risk of birth defects as a potential side effect. Such teratogenic pharmaceuticals currently listed in the Physicians' Desk Reference (Medical Economics Company, publ. 2000) include, for example, isotretinoin (ACCUTANE®), acitretin (SORIATANE®), finasteride (PROPECIA®), fluorouracil (FLUOROPLEX® or EFUDEX®), griseofulvin (GRIFULVIN®, FULVICIN®, GRIS-PEG®, GRISACTIN®), goserelin (ZOLADEX®), podophyllin/podophyllum (PODOCOM®), stanozolol (WINSTROL®), tazarotene (TAZORAC®), methotrexate and thalidomide (THALIDOMID®). Pharmaceutically or therapeutically effective amounts and dosages for such pharmaceuticals are known in the art (e.g., Physicians' Desk Reference, ibid.). Certain of these pharmaceuticals (the latter two, for example) are associated with teratogenicity in a pregnant female, even if the teratogen was taken not by the female, but by the male partner.

i) Thalidomide

Thalidomide, 2-(2,6-Dioxo-3-piperidinyl)-1H-isoindole-1,3(2H)-dione, was previously commercially available as a sedative and hypnotic. Its preparation is described in British Patent No. 768,821 (1957). It was commercially available under the tradenames DISTAVAL®, SOFTENON®, SEDALIS®, TALIMOL®, PANTOSEDIV®, NEUROSEDIV®. After being widely used, it was recognized as a teratogen. Fratta, I. D. et al., 7 Toxicol. Appl. Pharmacol. 268 (1965); Schumacher, H. et al., 160 J. Pharmacol. Exp. Ther. 189 (1968). The mechanism of teratogenic activity has subsequently been elucidated. Gordon, G. B. et al., 78 Proc. Nat. Acad. Sci. 2545 (1981). Its use as a sedative hypnotic was thus banned.

Thalidomide was subsequently investigated as an immunomodulator. McHugh, S. M. et al., 99 Clin. Exp. Immunol. 160 (1995). Thus, its in vitro activity against HIV-1 virus has been investigated, Makonkawkeyoon, S. et al., 90 Proc. Nat. Acad. Sci. 5974 (1993), as well as its clinical use against HIV-infection related aphthous ulceration, Paterson, D. L. et al., 20 Clin. Infect. Dis. 250 (1995). It has also been considered for a variety of other immunological disorders. Schuler, U. et al., 7 Drug Safety 116 (1992). It has been found successful and effective in treating leprosy, Crawford, C. L., 13 Adverse Drug React. Toxicol. Rev. 177 (1994). It is thus currently known as a selective inhibitor of tumor necrosis factor alpha. Thus, despite its dangerous side effect as a teratogen, it is a useful pharmaceutical.

Educational Combination Kit

One can achieve the benefits of my invention, yet alleviate entirely the risk of chemical cross-reactivity between the contraceptive pharmaceutical and the teratogen, by manufacturing an article of manufacture which includes the teratogen and the contraceptive packaged together in a unitary package for sale as an undivided unit. For example, the educational kit can be made with an outer package such as a box with a first chamber to receive a vial of teratogenic pharmaceutical and a second chamber to receive a contraceptive. The contraceptive can be a device (e.g., a condom or an intrauterine device), a container of birth control pharmaceuticals, or the like. I in fact prefer that two different contraceptives are included in the kit (e.g., an intrauterine device together with a container of birth control pills). Similarly, while I prefer that the outer package have more than one chamber, it is not necessary; a simple box or even vacuum-packaging will do, as long as it has both the teratogen and the contraceptive.

Note that this article of manufacture does not assure the contraceptive will in fact be used. I.e., the patient may discard or simply refuse to use the contraceptive, perhaps based on religious grounds. The article of manufacture does, however, serve as a clear educational device to remind the patient to avoid becoming pregnant while taking the teratogen.

Composition of Matter for Oral Administration

One can achieve the benefits of my invention by manufacturing a pharmaceutical composition of matter comprising a teratogenic pharmaceutical and a contraceptive pharmaceutical. For example, an acne medication may be formulated:

| | | |
|---|---|---|
| Isotretinoin | 40 | mg |
| Ethinyl estradiol | 20-30 | ìg |
| Gestodene | 75 | ìg |

The formulation is complete as desired, with butylated hydroxyanisole, edentate disodium, hydrogenated soybean oil flakes, hydrogenated vegetable oil, soybean oil and coloring agent. Stabilizer and antioxidant are included as needed to mitigate chemical cross-reaction between the teratogen and the contraceptive.

As another example, an oral anti-leprotic pharmaceutical composition may be formulated:

| | | |
|---|---|---|
| Thalidomide | 50-400 | mg |
| Estrogen | 20-30 | ìg |

The formulation is complete as desired, with anhydrous lactose, microcrystalline cellulose, polyvinylpyrrolidone, stearic acid, colloidal anhydrous silica and gelatin. As with the preceding example, stabilizer and antioxidant are useable as needed to mitigate chemical cross-reaction between the teratogen and the contraceptive.

ii) Dosage Amounts

With respect to the dosage amount of the pharmaceutical components used here, the specific dose may vary depending on the age and weight of the patient, the severity of the symptoms, the incidence of non-teratogenic side effects and the like. For isotretinoin, for example, it is known in the art to administer 5-40 milligrams of isotretinoin two times per day. Similarly, for a contraceptive, it is known in the art to use estrogen, 20 or more micrograms per day, or gestodene 75 micrograms per day together with 20-30 micrograms per day of ethinyl estradiol per day. Other currently-known contraceptive preparations are discussed in my Information Disclosure Statement at Shangold et al., U.S. Pat. No. 6,214,815.

I use the phrase “effective amount” of a pharmaceutical to mean the amount known in the art as effective for the disease indicated. For example, it is known in the art that thalidomide may function as a hypnotic and as a tumor necrosis factor inhibitor. The amount effective for the former indication, however, is different from the amount used for the latter. Effective amounts are, however, not difficult to ascertain; for each pharmaceutical drug substance sold in the United States, the “effective amount” to treat the labeled indication is specified by the Food & Drug Administration.

Article of Manufacture for Transdermal Drug Delivery

A trans-dermal skin patch using my combinations can achieve the same benefit of minimizing the risk of unwanted pregnancy. For example, one can prepare a trans-dermal skin patch as follows:

| | | |
|---|---|---|
| Progesterone | 300 | mg |
| Isotretinoin | 40-400 | mg |
| PDMS-382 (Dow Corning) pre-polymer | 9.2 | gm |
| 5-Amino-5-ethyl-2-(3-haptyl_-1,3-dioxane | 500 | mg |
| Polymerization initiator | 1 | drop |

After mixture, the mixture is poured into sheet molds and allowed to set at room temperature for 24 hours. After set-up is complete, the sheet may be cut into discs 1 centimeter in diameter. Other trans-dermal formulations are well within the teachings of the art, and depend on the nature of the specific skin penetrating agent used.

Claim Scope

In the claims, I use the singular (a, an, the, said) to include one or more. For example, “a contraceptive” means one or more contraceptives. I use the terms “label” and “labeling” as defined in the Federal Food, Drug & Cosmetic Act and the regulations promulgated thereunder. The term unit dose form means in a form wherein both components are intended to be taken together as one big pill or as two small pills together.

While I have described and illustrated my invention with reference to certain examples or preferred embodiments thereof, those skilled in the art will appreciate that various changes, modifications and substitutions can be made therein without departing from the spirit of my invention. For example, effective dosages other than the preferred ranges set forth above with respect to each pharmaceutical may be applicable as a consequence of variations of the responsiveness of the person treated, severity of the symptoms, dosage related adverse side effects, and similar considerations. Thus, I intend my invention to be limited only by the scope of my claims appended.

I claim:

1. An educational article of manufacture useful to increase patient awareness of the teratogenicity of a pharmaceutical, said article of manufacture comprising:
a teratogenic pharmaceutical packaged together with a contraceptive; and
labeling specifying avoidance of pregnancy while using said teratogenic pharmaceutical.

2. The article of manufacture of claim 1, wherein said potentially teratogenic pharmaceutical is isotretinoin.

3. The article of manufacture of claim 1, wherein said contraceptive comprises a device.

4. The article of manufacture of claim 3, wherein said device is a contraceptive intrauterine device.

5. The article of manufacture of claim 1, wherein said contraceptive is a pharmaceutical in an amount effective as a contraceptive.

6. A pharmaceutical composition of matter comprising:
a first pharmaceutical in an amount potentially teratogenic, and
a second pharmaceutical in an amount effective as a contraceptive,
said composition of matter in a unit dose form.

7. The composition of matter of claim 6, wherein said unit dose administration is trans-dermal administration.

8. The composition of matter of claim 6, wherein said unit dose administration is oral administration.

9. The composition of matter of claim 8, wherein said teratogenic pharmaceutical is isotretinoin.

10. The composition of matter of claim 9, wherein said isotretinoin is present in an amount of about 5 mg to about 40 mg per dose.
